(19) United States
(12) Patent Application Publication
(10) Pub. No.: US 2026/0091002 A1
(43) Pub. Date: Apr. 2, 2026

(54) COMPOSITION FOR ANTIMICROBIAL AND ANTIBIOFILM ACTIVITY CONTAINING HALOGENATED PHENOL DERIVATIVE AS ACTIVE INGREDIENT

(71) Applicant: Industry Academic Cooperation Foundation of Yeungnam University, Gyeongsan-si (KR)

(72) Inventors: Jintae LEE, Daegu (KR); Jin-Hyung LEE, Daegu (KR); OLANREWAJU ABDULRAUF OLALEKAN, Gyeongsan-si (KR)

(73) Assignee: Industry Academic Cooperation Foundation of Yeungnam University, Gyeongsan-si (KR)

(21) Appl. No.: 19/232,973

(22) Filed: Jun. 10, 2025

(30) Foreign Application Priority Data
Sep. 27, 2024 (KR) 10-2024-0131345

(51) Int. Cl. A61K 31/055; A61K 31/43; A61K 31/65; A61K 31/7036; A61K 38/14; A61P 31/04

(52) U.S. Cl. CPC A61K 31/055; A61K 31/43; A61K 31/65; A61K 31/7036; A61K 38/14; A61P 31/04

(57) ABSTRACT

The present disclosure relates to an antimicrobial and antibiofilm composition containing a halogenated phenol derivative as an active ingredient, and more specifically, to an antibiofilm composition against Staphylococcus aureus or pathogenic microorganisms, an antimicrobial composition, and a composition for treating an infection caused by a biofilm of Staphylococcus aureus or pathogenic microorganisms, containing a halogenated phenol derivative or a pharmaceutically acceptable salt thereof having excellent antimicrobial and antibiofilm activity against Staphylococcus aureus and/or pathogenic microorganisms as an active ingredient.

### CROSS-REFERENCE TO RELATED APPLICATION

This application claims the benefit of Korean Patent Application No. 10-2024-0131345 filed on Sep. 27, 2024, in the Korean Intellectual Property Office, the entire disclosure of which is incorporated herein by reference for all purposes.

### BACKGROUND OF THE INVENTION

The present disclosure relates to a compound having antimicrobial and antibiofilm activity, and more specifically, to an antimicrobial and biofilm formation-inhibiting composition containing a halogenated phenol derivative as an active ingredient.

A microbial biofilm, or a biofilm, is an adherent microbial colony that grows on surfaces by self-produced extracellular polymeric substances, and these films are ubiquitous in natural, medical, and engineering environments. Pathogenic biofilms can cause serious problems to human health since they are resistant to existing antibiotics, host defense systems, and external stresses to perpetuate chronic bacterial infections.

Most antibiotics aim to inhibit microbial growth, ideally without harming the host or the environment. However, overuse of these drugs has led to the emergence of drug-resistant pathogens worldwide. Bacterial and fungal biofilms play a crucial role in antimicrobial resistance and various device-related infections, with various pathogenic microorganisms forming biofilms that further increase their resistance to antibiotics. In other words, once the biofilm is formed, it is considered that it has become resistant to antibiotics, in which case, the bacterial sensitivity to antibiotics is reduced to make antibiotics hardly effective, and in particular, infections by bacteria forming a biofilm are often caused by multi-resistant bacteria that are resistant to multiple antibiotics, making the problem even more serious.

Staphylococcus aureus, which is a causative bacterium of a variety of acute and chronic infections, often shows antibiotic-resistance and is attributable to outbreaks of nosocomial infections worldwide. While antibiotics used for infections by Staphylococcus aureus are penicillin, carbapenem, cephalosporin, and beta-lactam series, due to the abuse of antibiotics, penicillin resistance has been around for a long time with global issues concerning the increase in methicillin-resistant Staphylococcus aureus (MRSA) and vancomycin-resistant Staphylococcus aureus (VRSA), and antibiotic prescriptions are not effective in cases of MRSA or VRSA infection, thereby posing a serious threat to life.

Additionally, Staphylococcus aureus secretes several exotoxins, including α-hemolysin, enterotoxins, coagulase, and protein A. In particular, α-hemolysin (also known as α-toxin) is a major virulence factor and associated with a pathogen causing sepsis, pneumonia, and severe skin infections and known to contribute to biofilm formation.

Therefore, there is a need for non-toxic compounds that are non-drug resistant and capable of inhibiting biofilms of Staphylococcus aureus.

### SUMMARY OF THE INVENTION

An object of the present disclosure is to provide a compound having excellent antimicrobial and antibiofilm activity.

Another object of the present disclosure is to provide an antibiofilm coating composition including the compound.

Another object of the present disclosure is to provide a composition for treating an infection caused by a biofilm, including the compound.

To achieve the above objects, the present disclosure provides an antibiofilm composition against Staphylococcus aureus or pathogenic microorganisms, including a compound represented by the following Chemical Formula 1 or a pharmaceutically acceptable salt thereof as an active ingredient:

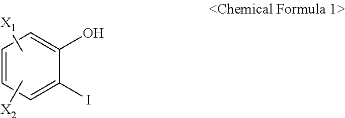

wherein, in the formula,
X₁ and X₂ may be the same or different and each independently selected from hydrogen or halogen.

The present disclosure provides an antibiofilm coating composition against Staphylococcus aureus or pathogenic microorganisms, including the composition.

The present disclosure provides an antimicrobial composition against Staphylococcus aureus or pathogenic microorganisms, including the compound represented by Chemical Formula 1 or a pharmaceutically acceptable salt thereof as an active ingredient.

The present disclosure provides a pharmaceutical composition for preventing or treating an infection caused by a biofilm of Staphylococcus aureus or pathogenic microorganisms, including the compound represented by Chemical Formula 1 or a pharmaceutically acceptable salt thereof as an active ingredient.

In addition, the present disclosure provides a health functional food composition for preventing or ameliorating an infection caused by a biofilm of Staphylococcus aureus or pathogenic microorganisms, including the compound represented by Chemical Formula 1 or a pharmaceutically acceptable salt thereof as an active ingredient.

A halogenated phenol derivative compound according to the present disclosure has excellent antimicrobial and antibiofilm activity against Staphylococcus aureus and/or pathogenic microorganisms.

The compound according to the present disclosure has low cytotoxicity while inhibiting major virulence factors such as hemolysin and proteases from Staphylococcus aureus, and thus may be utilized as an antimicrobial and antibiofilm composition against the factors.

Moreover, the compound according to the present disclosure may be utilized as a composition for treating various infectious diseases caused by a biofilm of Staphylococcus aureus and/or pathogenic microorganisms.

### BRIEF DESCRIPTION OF THE DRAWINGS

FIG. 1 shows analysis of inhibitory activity on biofilm formation by Staphylococcus aureus following treatment with various phenol compounds, wherein, specifically, FIG. 1A shows a comparison of antibiofilm activity of control antibiotics (vancomycin, gentamicin) and 2,4,6-triiodophenol (2,4,6-TIP), FIGS. 1B to 1F show results of analyzing inhibitory activity on biofilm formation by a Staphylococcus aureus strain (ATCC 6538) when treated with 2,4,6-triiodophenol (2,4,6-TIP), 5-chloro-2-iodophenol, 5-bromo-2-iodophenol, 4-bromo-2-iodophenol, and 4-chloro-2-iodophenol, respectively (n=2 biologically independent samples, error bars represent standard deviation, * P<0.05 vs. untreated control group), FIGS. 1G and 1F show results of analyzing changes in cell growth of Staphylococcus aureus (MSSA 6538) and methicillin-resistant Staphylococcus aureus (MRSA MW2) strains when treated with 2,4,6-TIP with the best activity, and FIG. 1I shows a result of analyzing the cell viability (colony-forming unit: CFU) of Staphylococcus aureus (MSSA 6538) when treated with 2,4,6-TIP.

FIG. 2 shows analysis of antimicrobial and antibiofilm activity against various microorganisms when treated with 2,4,6-TIP in a 96-well plate, wherein FIGS. 2A to 2F show analysis of activity against methicillin-resistant Staphylococcus aureus (MRSA MW2), Staphylococcus epidermidis (S. epidermidis), drug-resistant Candida albicans (C. albicans), Vibrio parahaemolyticus (V. parahaemolyticus), uropathogenic E. coli, and two microbial models (Staphylococcus aureus MSSA 6538+Candida albicans), respectively (n=2 biologically independent samples, error bars represent standard deviations.* P<0.05 vs. untreated control group).

FIG. 3 shows results of analyzing inhibitory activity of 2,4,6-TIP on biofilm formation by Staphylococcus aureus MSSA 6538, wherein FIG. 3A shows images of biofilms analyzed in 2D and 3D via the iRiS™ digital cell imaging system, and FIG. 3B shows results of analyzing the inhibitory activity of 2,4,6-TIP on biofilm formation by Staphylococcus aureus on a nylon surface using a scanning electron microscope (SEM). The black scale bar represents 50 μm, the red scale bar represents 5 μm, two or more independent cultures were performed for microscopic experiments, and 10 or more random locations were analyzed.

FIG. 4 shows results of analyzing inhibitory activity of 2,4,6-TIP on biofilm formation by a model combined with two types of microorganisms (Staphylococcus aureus MSSA 6538+Candida albicans), wherein FIG. 4A shows images of two types of biofilms analyzed in 2D and 3D using the iRiS™ digital cell imaging system, and FIG. 4B shows results of analyzing inhibitory activity of 2,4,6-TIP on biofilm formation by the two types on a nylon surface using a scanning electron microscope (SEM). The large cells are Candida albicans cells, and the small round cells are Staphylococcus aureus cells. The black scale bar represents 50 μm, the yellow scale bar represents 10 μm, two or more independent cultures were performed for microscopic experiments, and 10 or more random locations were analyzed.

FIG. 5 shows results of analyzing inhibitory activity of 2,4,6-TIP on hemolysin and protease, which are virulence factors of Staphylococcus aureus (* P<0.05 vs. untreated control group).

FIG. 6 shows results of analyzing a compound toxicity of phenol and 2,4,6-TIP in plant (cabbage) germination and nematode models (* P<0.05 vs. untreated control group).

### DETAILED DESCRIPTION

Hereinafter, the present disclosure will be described in detail.

Although phenol is generally known for its toxicity and corrosiveness, it has been studied recently that halogenation enhances drug affinity, improves an enzyme-catalytic activity, and increases target protein binding and thermal stability. Under this background, the inventors of the present disclosure have completed the present disclosure by determining that a halogenated phenol derivative compound, particularly 2,4,6-triiodophenol (2,4,6-TIP), has an excellent antibiofilm and antimicrobial activity against Staphylococcus aureus, other pathogenic microorganisms, or a combination of these microorganisms.

The present disclosure provides an antimicrobial or antibiofilm composition including a halogenated phenol derivative compound as an active ingredient.

Preferably, the present disclosure provides an antibiofilm composition against Staphylococcus aureus or pathogenic microorganisms, including a compound represented by the following Chemical Formula 1 or a pharmaceutically acceptable salt thereof as an active ingredient:

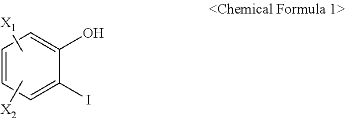

wherein, in the formula, X₁ and X₂ may be the same or different and each independently selected from hydrogen or halogen.

The compound may include, but is not limited to, 2,4,6-triiodophenol (2,4,6-TIP), 5-chloro-2-iodophenol, 5-bromo-2-iodophenol, 4-bromo-2-iodophenol, and 4-chloro-2-iodophenol.

More preferably, the compound may be 2,4,6-triiodophenol (2,4,6-TIP) represented by the following Chemical Formula 2, but is not limited thereto.

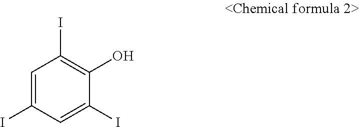

As used herein, the term “pharmaceutically or foodologically acceptable” refers to a salt that is non-toxic to cells or humans exposed to the composition and has a safety and efficacy profile suitable for administration to humans.

The salt may be used in any one form of a basic salt or an acidic salt that is pharmaceutically or foodologically acceptable. The basic salt may be used in any one form of organic or inorganic basic salts and selected from the group consisting of sodium salts, potassium salts, calcium salts, lithium salts, magnesium salts, cesium salts, aminium salt, ammonium salts, triethylamine salts, and pyridinium salts. Acid addition salt that is formed by free acid is useful as the acidic salt. As the free acid, inorganic acids and organic acids may be used, hydrochloric acid, bromic acid, sulfuric acid, sulfurous acid, phosphoric acid, diphosphoric acid, and nitric acid may be used as the inorganic acid, whereas citric acid, acetic acid, maleic acid, malic acid, fumaric acid, gluconic acid, methanesulfonic acid, benzenesulfonic acid, camphorsulfonic acid, oxalic acid, malonic acid, glutaric acid, acetic acid, glycolic acid, succinic acid, tartaric acid, 4-toluenesulfonic acid, galacturonic acid, embonic acid, glutamic acid, citric acid, aspartic acid, and stearic acid may be used as the organic acid, and salts formed using various inorganic acids and organic acids that are commonly used in the art may all be included without limitation.

In addition, the composition may include not only the salt described above, but also all salts, hydrates, solvates, and derivatives, which may be prepared by conventional methods. The addition salt may be prepared by conventional methods and it may be prepared by dissolving the compound in a water-miscible organic solvent, e.g., acetone, methanol, ethanol, or acetonitrile and then carrying out precipitation or crystallization after adding an excess of organic bases or an aqueous basic solution of inorganic bases. Alternatively, preparation may be carried out by obtaining the addition salt by evaporation of the solvent or excess base from the mixture followed by drying or suction filtration of the precipitated salts.

The compound or salt thereof may be included in the composition at a concentration of 5 to 500 μg/mL.

In the antibiofilm composition according to the present disclosure, the Staphylococcus aureus may be any one or more selected from the group consisting of Staphylococcus aureus, methicillin-sensitive Staphylococcus aureus (MSSA), methicillin-resistant Staphylococcus aureus (MRSA), and vancomycin-resistant Staphylococcus aureus (VRSA), but is not limited thereto.

The pathogenic microorganism may be selected from the group consisting of Staphylococcus epidermidis, drug-resistant Candida albicans, uropathogenic Escherichia coli, Vibrio parahaemolyticus, and two or more types of microorganisms combined with those and Staphylococcus aureus, but is not limited thereto.

The composition may inhibit a virulence factor of Staphylococcus aureus, and the virulence factor may include hemolysin or protease.

The composition may further include antibiotics, specifically, but not limited to, any one or more antibiotics selected from the group consisting of vancomycin, gentamycin, streptomycin, ampicillin, and oxytetracycline.

The present disclosure provides an antibiofilm coating composition against Staphylococcus aureus or pathogenic microorganisms, including the antibiofilm composition.

The coating composition may be utilized as a coating composition to be coated on a food manufacturing machine, a food packaging container, a medical device, a medical material, or a medical implant.

The present disclosure provides an antimicrobial composition against Staphylococcus aureus or pathogenic microorganisms, including a compound represented by the following Chemical Formula 1 or a pharmaceutically acceptable salt thereof as an active ingredient:

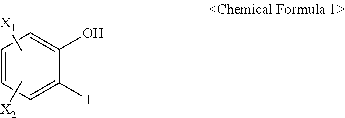

wherein, in the formula, X₁ and X₂ may be the same or different and each independently selected from hydrogen or halogen.

Preferably, the present disclosure provides an antimicrobial composition against Staphylococcus aureus or pathogenic microorganisms, including 2,4,6-triiodophenol (2,4,6-TIP) represented by the following Chemical Formula 2 as an active ingredient:

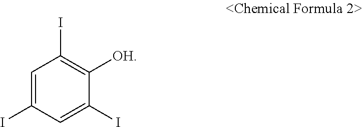

The composition may have an excellent antimicrobial activity against any one or more Staphylococcus aureus selected from the group consisting of Staphylococcus aureus, methicillin-sensitive Staphylococcus aureus (MSSA), methicillin-resistant Staphylococcus aureus (MRSA), and vancomycin-resistant Staphylococcus aureus (VRSA); or pathogenic microorganisms selected from the group consisting of Staphylococcus epidermidis, drug-resistant Candida albicans, uropathogenic Escherichia coli, Vibrio parahaemolyticus, and two or more types of microorganisms combined with those and Staphylococcus aureus.

The present disclosure provides a pharmaceutical composition for preventing or treating an infection caused by a biofilm of Staphylococcus aureus or pathogenic microorganisms, including a compound represented by the following Chemical Formula 1 or a pharmaceutically acceptable salt thereof as an active ingredient:

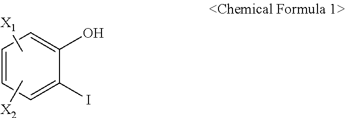

wherein, in the formula, X₁ and X₂ may be the same or different and each independently selected from hydrogen or halogen.

Preferably, the present disclosure provides a pharmaceutical composition for preventing or treating an infection caused by a biofilm of Staphylococcus aureus or pathogenic microorganisms, including 2,4,6-triiodophenol (2,4,6-TIP) represented by the following Chemical Formula 2 as an active ingredient:

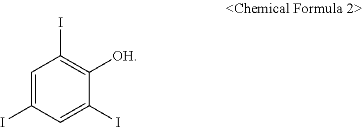

The infection caused by the biofilm of Staphylococcus aureus or pathogenic microorganisms may be any one or more selected from the group consisting of soft tissue infection (cellulitis, pyomyositis), septic arthritis, suppurative osteomyelitis, otitis media, pneumonia, postoperative wound infection, bacteremia, endocarditis, sepsis, food poisoning, enteritis, dermatitis, vaginitis, nephritis, cystitis, urethritis, prostatitis, inflammatory diseases caused by urinary catheters or intravenous catheters, and nosocomial infections, but is not limited thereto.

Staphylococcus aureus is one of the most widely distributed bacteria in nature, found as a colony in the nasal cavity, pharynx, skin, and hair of more than 30% of healthy people. The bacterium is known to be the causative bacterium of soft tissue infections (cellulitis, pyomyositis), septic arthritis, pyogenic osteomyelitis, otitis media, pneumonia, postoperative wound infections, bacteremia, endocarditis, and food poisoning. In particular, methicillin-resistant Staphylococcus aureus is a major cause of hospital-acquired infections, causing infections as it enters the bloodstream through the venous catheter of hospitalized patients.

Staphylococcus epidermidis causes biofilms to grow on medical devices placed in the body, most commonly on intravenous catheters and medical prosthetics, but the biofilm may also develop in patients undergoing dialysis or in people with contaminated medical devices implanted. It also the most commonly causes endocarditis in patients with defective heart valves and is a causative bacterium of sepsis

Uropathogenic Escherichia coli is the causative bacterium of most urinary tract infections and invades the urinary system, including kidneys, ureters, bladder, urethra, and prostate to cause inflammatory reactions, Vibrio parahaemolyticus is the main causative bacterium of food poisoning, and Candida albicans is the most common causative bacterium of fungal infections and may cause dermatitis and vaginitis depending on the site of infection. The pharmaceutical composition according to the present disclosure may be prepared according to a conventional method in the pharmaceutical field. The pharmaceutical composition may be combined with an appropriate pharmaceutically acceptable carrier depending on the formulation, and may be manufactured by further including, if necessary, excipients, diluents, dispersants, emulsifiers, buffers, stabilizers, binders, disintegrants, and solvents. The appropriate carriers, which do not degrade the activity and properties of the compound according to the present disclosure or pharmaceutically acceptable salt thereof, may be selected differently depending on the dosage form and formulation.

The pharmaceutical composition may be applied in any dosage form, and more specifically, it may be formulated to be used as oral formulations and parental formulations of an external preparation, a suppository, and a sterile injection solution according to a conventional method.

Among the oral formulations, the solid formulation is in the form of tablets, pills, acids, granules, and capsules and may be prepared by mixing at least one or more excipients, such as starch, calcium carbonate, sucrose, lactose, sorbitol, mannitol, cellulose, and gelatin, and in addition to simple excipients, lubricants such as magnesium stearate and talc may also be included. Additionally, in the case of capsule formulations, a liquid carrier such as fatty oil may be further included in addition to the above-mentioned substances.

Among the oral formulations, liquid formulation includes suspensions, solutions, emulsions, and syrups, and in addition to commonly used simple diluents such as water and liquid paraffin, various excipients such as wetting agents, sweeteners, fragrances, and preservatives may be included.

The parenteral formulations may include sterile aqueous solutions, non-aqueous solvents, suspensions, emulsions, lyophilized preparations, and suppositories. Propylene glycol, polyethylene glycol, vegetable oils such as olive oil, and injectable esters such as ethyl oleate may be used as non-aqueous solvents and suspensions. As for base materials of suppositories, witepsol, macrogol, tween 61, cacao butter, laurin fat, and glycerogelatin may be used. Any suitable preparation known in the art may be used without limitation.

In the pharmaceutical composition according to the present disclosure, the pharmaceutical composition may be administered in a pharmaceutically effective amount.

As used herein, the term “pharmaceutically effective amount” refers to an amount sufficient to treat a disease at a reasonable benefit/risk ratio applicable to medical treatment without causing adverse effects.

The effective dosage level of the pharmaceutical composition may be determined differently depending on elements including the intended use, age, sex, weight and health status of a patient, type of the disease, severity, activity of the drug, sensitivity to the drug, method of administration, time of administration, route and excretion rate, duration of treatment, drugs used in combination or concurrently, and other elements well known in the other medical fields. For example, although not constant, it may be administered generally from 0.001 to 1000 mg/kg, preferably from 0.01 to 100 mg/kg, once or several times daily. The dosage does not limit the scope of the present disclosure in any way.

The pharmaceutical composition may be administered to any animal with probability of developing an infection caused by a biofilm of Staphylococcus aureus or pathogenic microorganisms, and the animal may include, for example, humans and primates, as well as livestock such as cows, pigs, horses, and dogs.

The pharmaceutical composition may be administered through an appropriate administration route depending on the formulation form, and may be administered through various routes, either oral or parenteral, as long as it may reach the target tissue. The method of administration is not particularly limited and may be administered by conventional methods such as oral, rectal or intravenous, intramuscular, skin application, respiratory inhalation, intrauterine epidural, or intracerebroventricular injection.

The pharmaceutical composition may be used alone for the prevention or treatment of infections caused by a biofilm of Staphylococcus aureus or pathogenic microorganisms or in combination with surgery or other drug treatments.

In addition, the present disclosure provides a health functional food composition for preventing or ameliorating an infection caused by a biofilm of Staphylococcus aureus or pathogenic microorganisms, including a compound represented by the following Chemical Formula 1 or a foodologically acceptable salt thereof as an active ingredient:

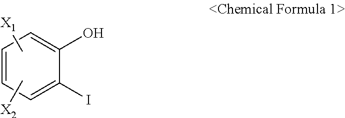

wherein, in the formula, X₁ and X₂ may be the same or different and each independently selected from hydrogen or halogen.

Preferably, the present disclosure provides a health functional food composition for preventing or ameliorating an infection caused by a biofilm of Staphylococcus aureus or pathogenic microorganisms, including 2,4,6-triiodophenol (2,4,6-TIP) represented by the following Chemical Formula 2 as an active ingredient:

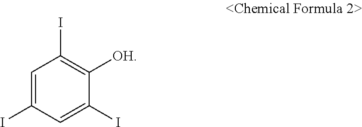

The corresponding features may be replaced by those described above.

In the health functional food composition according to the present disclosure, the health functional food may be manufactured in the form of powder, granules, tablets, capsules, syrup, or beverage, for the purpose of prevention or amelioration of infections caused by a biofilm of Staphylococcus aureus or pathogenic microorganisms. There is no limitation on the form that the health functional food may take, and it may be formulated in the same manner as the pharmaceutical composition to be used as a functional food or added to various foods.

The health functional food may include all foods in the conventional sense. For example, beverages and various drinks, fruits and processed foods thereof (canned fruits, jams, etc.), fish and meat and processed foods thereof (ham, bacon, etc.), bread and noodles, cookies and snacks, and dairy products (butter, cheese, etc.) are possible, and all functional foods in the conventional sense may be included. It may also include food used as feed for animals.

The health functional food composition according to the present disclosure may be manufactured by further including food additives (food additive substances) that are commonly used in the art and foodologically acceptable and other appropriate auxiliary ingredients. Unless otherwise specified, suitability as a food additive may be determined by the standards and criteria related to corresponding items according to the general rules and general test methods of Korean Food Additives Codex approved by the Ministry of Food and Drug Safety. The items listed in the “Korean Food Additives Codex” may include, for example, chemically synthesized compounds such as ketones, glycine, potassium citrate, nicotinic acid, and cinnamic acid; natural additives such as persimmon color, licorice extracts, crystallized cellulose, kaoliang color, and guar gum; and mixed preparations such as sodium L-glutamate preparations, noodle-added alkali agents, preservative agents, and tar color agents.

The other auxiliary ingredients may additionally include, for example, flavoring agents, natural carbohydrates, sweeteners, vitamins, electrolytes, coloring agents, pectic acid, alginic acid, organic acids, protective colloid thickeners, pH adjusters, stabilizers, preservatives, glycerin, alcohol, and carbonating agents. In particular, monosaccharides such as glucose and fructose, disaccharides such as maltose and sucrose, polysaccharides such as dextrin and cyclodextrin, and sugar alcohols such as xylitol, sorbitol and erythritol may be used as the natural carbohydrates, and natural sweeteners, such as thaumatin and stevia extract, and synthetic sweeteners, such as saccharin and aspartame, may be used as the sweeteners.

The effective dose of the compound or foodologically acceptable salt thereof contained in the health functional food according to the present disclosure may be appropriately adjusted depending on the intended use, such as prevention or amelioration of an infection caused by a biofilm of Staphylococcus aureus or pathogenic microorganisms.

The health functional food composition has the advantage of having no side effects that may occur with long-term intake of general medicines by containing food as a raw material and is highly portable, so it may be taken as a supplement for preventing or ameliorating infections caused by a biofilm of Staphylococcus aureus or pathogenic microorganisms.

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### <Experimental Example 1> Analysis of Antimicrobial and Antibiofilm Activity of Halogenated Phenol Compounds

1. Preparation of microorganisms, culture materials, and compounds

In this Experimental Example, Staphylococcus aureus MSSA (ATCC 6538), methicillin-resistant Staphylococcus aureus MRSA MW2 (ATCC BAA-1707), Staphylococcus epidermidis (ATCC 14990), uropathogenic Escherichia coli O6:H1 CFT073 (ATCC 700928), Vibrio parahaemolyticus (ATCC 17802), drug-resistant Candida albicans DAY 185 (KCCM 51280), and two microbial models [Staphylococcus aureus+Candida albicans] were used for the microorganism.

All media [Luria-Bertani (LB), tryptic soy broth (TSB), potato dextrose broth (PDB), nutrient broth, and agar] were purchased from Becton Dickinson (Franklin Lakes, NJ, USA).

Phenol and five types of halogenated phenol compounds [2,4,6-triiodophenol (2,4,6-TIP), 5-chloro-2-iodophenol, 5-bromo-2-iodophenol, 4-bromo-2-iodophenol, and 4-chloro-2-iodophenol] were purchased from Combi-Blocks Inc. (San Diego, CA, USA). Vancomycin and gentamycin, antibiotics used as control, were purchased from Sigma-Aldrich.

All compounds were dissolved using dimethyl sulfoxide (DMSO), and 0.1% (v/v) DMSO was used as a negative control in the experiment. Of note, this concentration did not affect bacterial growth or biofilm formation.

### 2. Analysis of the Antimicrobial Activity

The minimum inhibitory concentration (MIC) was determined according to the Clinical Laboratory Standards Institute (CLSI) for bacteria and yeast.

Specifically, Staphylococcus aureus and other pathogenic microorganisms were cultured in 96-well polystyrene plates at 37° C. for 24 hours in the presence or absence of the phenol and five types of halogenated phenol compounds, and then the culture turbidity was measured at 600 nm using a spectrophotometer (Optizen 2120 UV; Mecasys Co. Ltd., Daejeon, Republic of Korea). The minimum inhibitory concentration of the compound was set if there is no cell growth compared to the initial cell concentration. All experiments were performed through at least two independent cultures.

### 3. Biofilm Assay in 96-Well Plates

Biofilms of various microorganisms were formed in 96-well polystyrene plates as previously described. Briefly, for bacteria, the culture was performed overnight at an initial turbidity of OD 0.05 (˜2×10⁷ CFU/mL), and for Candida, inoculation was conducted into appropriate culture medium in a final volume of 300 μL with or without fatty acids at an initial turbidity of OD 0.1 (˜2×10⁵ CFU/mL) at 600 nm followed by culture at 37° C. for 24 hours without shaking. Biofilm cells attached to 96-well plates (SPL Life Sciences, Pocheon, Korea) were stained with 0.1% crystal violet from Sigma-Aldrich (St. Louis, USA) for 20 minutes, washed repeatedly with sterile distilled water, and resuspended in 95% ethanol. Plates were read at 570 nm, results of which were obtained from at least six replicates of two independent cultures. The percentage of inhibition ratio was expressed as the relative biofilm value (a compound treated biofilm/a compound untreated control×100).

### 4. Statistical Analysis

Replication numbers for the analyses are provided above and results are presented as means±SDs. Statistical analysis was performed using one-way ANOVA followed by Dunnett's test using SPSS version 23 (SPSS Inc., Chicago, USA). A P value less than 0.05 was considered significant, and an asterisk indicates a significant difference between treated and untreated samples.

As a result of analyzing the antimicrobial activity of phenol and five types of halogenated phenol compounds against Staphylococcus aureus (MSSA 6538), as shown in Table 1 below, 2,4,6-TIP had an MIC of 5 μg/mL, the other four types of halogenated phenol compounds had MIC of 50 μg/mL, and phenol had an MIC of 1000 μg/mL or higher.

Table 1 below shows compounds used in this Experimental Example and their minimum inhibitory concentrations.

TABLE 1

| | | | MIC |
|---|---|---|---|
| Chemical name | Structure | CAS Number | (μg/mL) |
| Phenol | 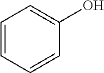 | 108-95-2 | >1000 |
| 2,4,6- Triiodophenol | 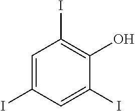 | 609-23-4 | 5 |
| 5-Chloro- 2-iodophenol | 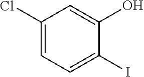 | 136808-72-5 | 50 |
| 5-Bromo- 2-iodophenol | 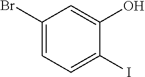 | 858855-11-5 | 50 |
| 4-Bromo- 2-iodophenol | 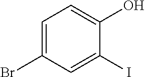 | 207115-22-8 | 50 |
| 4-Chloro- 2-iodophenol | 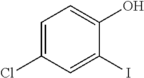 | 71643-66-8 | 50 |

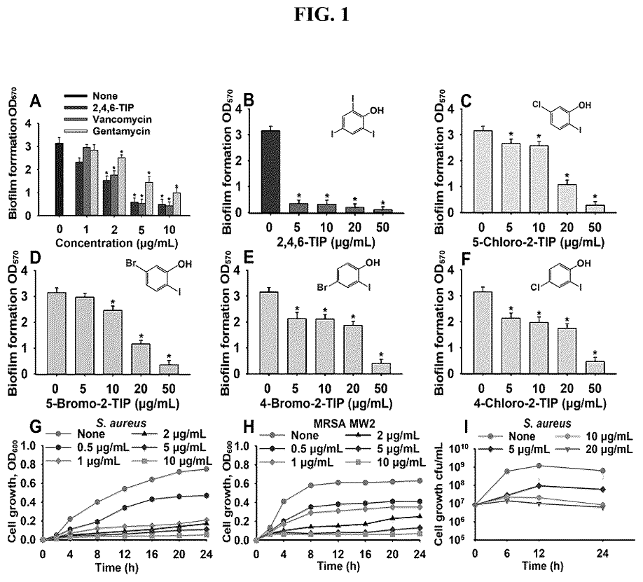

In addition, as a result of analyzing the antibiofilm activity of five types of halogenated phenol compounds against Staphylococcus aureus (MSSA 6538), 2,4,6-TIP showed similar antibiofilm activity compared to vancomycin and gentamycin, which are antibiotics used as controls, as shown in FIG. 1A. Five types of halogenated phenol compounds exhibited antibiofilm activity in a concentration-dependent manner (FIGS. 1B to 1F), and in particular, 2,4,6-TIP was shown to inhibit biofilm formation by 95% or greater even at a concentration of 5 μg/mL, revealing that among the compounds used in the above Experimental Examples, 2,4,6-TIP had the best activity of inhibiting biofilm of Staphylococcus aureus.

Accordingly, additional experiments were conducted on 2,4,6-TIP, which showed the best antimicrobial and antibiofilm activity.

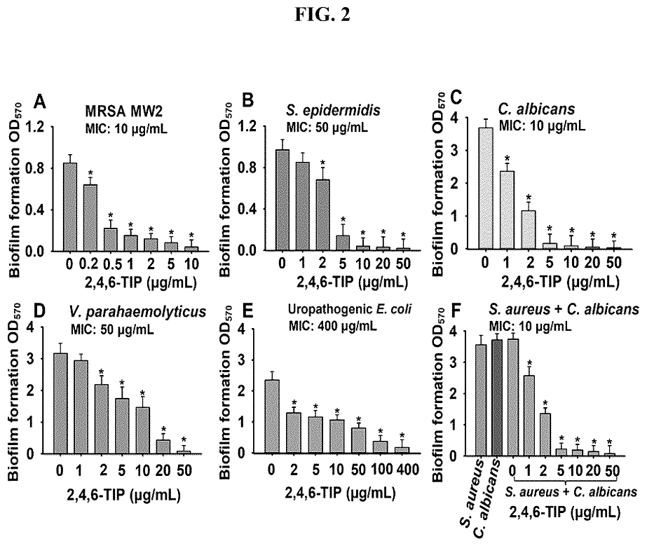

To identify the antimicrobial and antibiofilm activity of 2,4,6-TIP against drug-resistant Staphylococcus aureus and other pathogenic microorganisms, as a result of conducting experiments using the previously proceeded method, as shown in Table 2 and FIG. 2, 2,4,6-TIP exhibited a concentration-dependent antibiofilm activity against methicillin-resistant Staphylococcus aureus (MRSA MW2), Staphylococcus epidermidis (S. epidermidis), drug-resistant Candida albicans (C. albicans), Vibrio parahaemolyticus (V. parahaemolyticus), uropathogenic E. coli, and two types of microbial models (Staphylococcus aureus MSSA 6538+Candida albicans). In particular, best inhibitory activity was shown against methicillin-resistant Staphylococcus aureus and Candida albicans with MIC of 10 μg/mL, followed by Staphylococcus epidermidis, Vibrio parahaemolyticus, and uropathogenic E. coli in order.

In addition, since Staphylococcus aureus and Candida albicans are skin-inhabiting symbiotic strains that increase antimicrobial resistance, experiments were conducted on the two strains. As shown in FIG. 2F, 2,4,6-TIP had an MIC at concentration of 10 μg/mL for the two mixed strains and reduced composite biofilm formation by 95% at that concentration.

Table 2 below shows the minimum inhibitory concentrations of 2,4,6-triiodophenol (2,4,6-TIP) in various strains and culture media used in this Experimental Example.

TABLE 2

| | | | MIC (μg/mL) | |
|---|---|---|---|---|
| | | | 2,4,6-Triiodophenol | Inoculum size |
| Strains | Sources | Medium | (2,4,6-TIP) | (cfu/mL) |
| Staphylococcus aureus | ATCC 6538 | LB (Luria-Bertani) | 5 | ~2 × 10⁷ |
| MSSA | | | | |
| Staphylococcus aureus | ATCC BAA-1707 | LB + 0.2% glucose | 10 | ~2 × 10⁷ |
| MRSA MW2 | | | | |
| Staphylococcus | ATCC 14990 | LB + 0.2% glucose | 50 | ~2 × 10⁷ |
| epidermidis | | | | |
| Uropathogenic | ATCC 700928 | Nutrient Broth (NB) | 400 | ~2 × 10⁷ |
| Escherichia coli | | | | |
| O6: H1 CFT073 | | | | |
| Vibrio parahaemolyticus | ATCC 17802 | LB enriched with 3% | 50 | ~2 × 10⁷ |
| | | (w/v) NaCl (mLB) | | |
| Candida albicans | KCCM 51280 | Potato Dextrose Broth | 10 | ~2 × 10⁵ |
| DAY 185 | | (PDB). | | |
| S. aureus + C. albicans | ATCC 6538, | LB + POB | 10 | ~2 × 10⁷ |
| (Dual) | KCCM 51280 | | | ~2 × 10⁵ |

### <Experimental Example 2> Analysis of Antibiofilm Activity of 2,4,6-TIP Using Microscope

Single or double biofilms were formed at 37° C. for 24 hours without agitation under conditions with or without compounds as described in Experimental Example 1, No. 3, which were then subjected to gentle washing of floating cells three times with distilled water for removal and observed with live imaging microscopy using an iRiS™ digital cell imaging system (Logos BioSystems). Biofilm images were generated as color-coded 2D and 3D photographs using ImageJ.

Specifically, to identify the antibiofilm activity, cells in the Staphylococcus aureus biofilm formed on the nylon membrane were observed using a scanning electron microscope (SEM). Nylon membranes were cut to a size of 0.4×0.4 cm, and Staphylococcus aureus was cultured by adding the nylon membranes at 37° C. for 24 hours with or without the addition of 2,4,6-TIP (0, 2, 5, and 10 μg/mL). Cells attached to the nylon membrane were fixed with 2.5% glutaraldehyde and 2% formaldehyde and dehydrated using ethanol (50%, 70%, 80%, 90%, 95%, and 100%). After drying at a critical point, cells were sputter-coated with palladium/gold and observed under an S-4800 scanning electron microscope (Hitachi, Tokyo, Japan) at 15 kV.

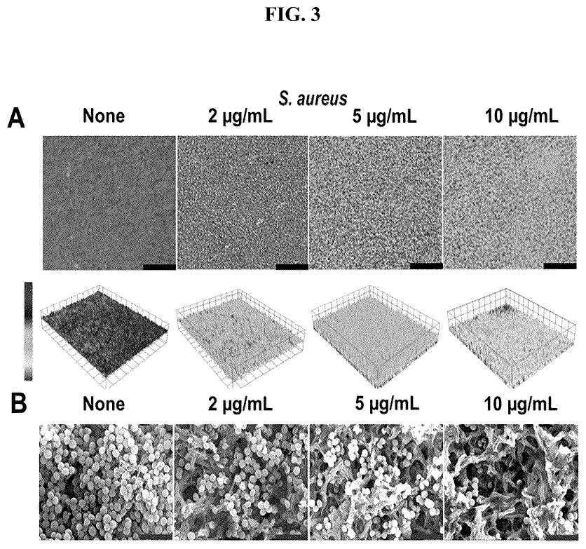

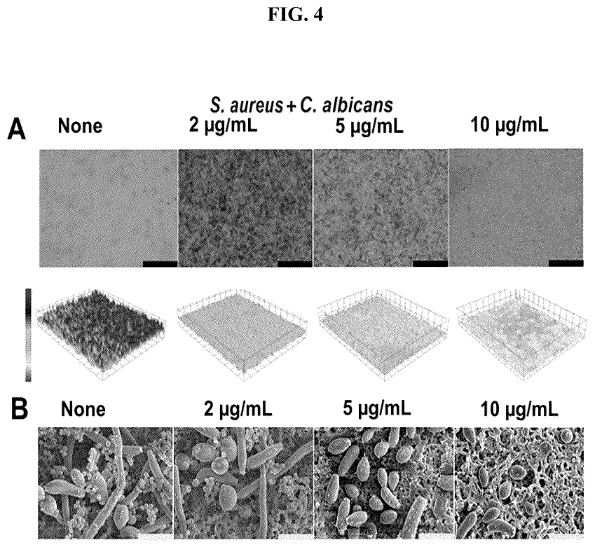

As a result of analyzing the antibiofilm activity of 2,4,6-TIP against Staphylococcus aureus using an optical microscope and a scanning electron microscope, as shown in FIGS. 3A and 3B, 2,4,6-TIP dose-dependently reduced the formation of Staphylococcus aureus biofilm, and as shown in FIGS. 4A and 4B, 2,4,6-TIP dose-dependently reduced the formation of mixed biofilms of Staphylococcus aureus and Candida albicans. As shown in the scanning electron microscopic images, it was found that 2,4,6-TIP reduced the cell count of Staphylococcus aureus and decreased the hyphae formation of Candida albicans.

### <Experimental Example 3> Analysis of Inhibitory Activity of 2,4,6-TIP on Staphylococcus aureus Virulence Factors

Staphylococcus aureus produces several virulence factors, such as hemolysins and proteases. Therefore, to determine the inhibitory activity of 2,4,6-TIP against Staphylococcus aureus virulence factors, hemolytic and protease activity was analyzed.

Hemolysis assay was conducted using fresh sheep blood purchased from MB cells. Red blood cells were separated by centrifuging sheep blood at 3000×g for 5 minutes, washed three times with PBS, and then diluted (330 μL of red blood cells/10 mL of PBS buffer). Staphylococcus aureus MSSA 6538 at an initial cell concentration of 1×10⁷ CFU/mL was added to 2 mL of fresh LB medium and 2,4,6-TIP (0, 0.2, 0.5, 1, 2, 5, and 10 μg/mL) and cultured at 37° C. with stirring at 250 rpm. The cell culture was added to diluted red blood cells, and the mixture of blood and Staphylococcus aureus (200 μL of cell culture) was reacted at 37° C. and 250 rpm for 3 hours to determine hemolysis. The culture was centrifuged at 16,600×g for 10 minutes, the supernatant was separated, and the optical density was measured at 543 nm.

For protease assay, Staphylococcus aureus MSSA 6538 at an initial cell concentration of 1×10⁷ CFU/mL was diluted (1:100) in fresh LB medium and treated with 2,4,6-TIP (0, 0.2, 0.5, 1, 2, 5, and 10 μg/mL). The culture was cultured at 37° C. for 24 hours with stirring at 250 rpm and then centrifuged at 12,000 rpm for 15 minutes to obtain the culture supernatant. Next, 75 μL of the culture supernatant was mixed with 125 μL of azocasein (2%), followed by culture at 37° C. for 30 minutes. After incubation, protein degradation was stopped by adding 600 μL of 10% trichloroacetic acid. The reaction tube was kept at −20° C. for 30 minutes to precipitate unreacted azocasein and then centrifuged at 10,000 rpm for 10 minutes to remove the precipitate. Afterwards, 600 μL of the supernatant and 700 μL of NaOH were mixed, and the absorbance was measured at 440 nm to quantify the protease activity.

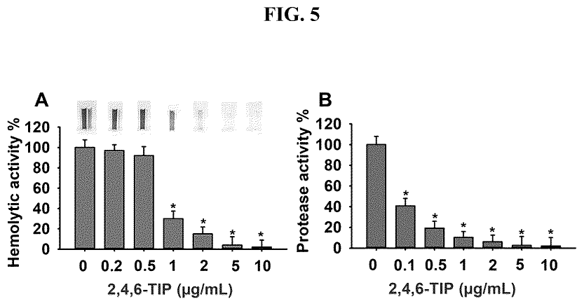

As a result of analyzing the inhibitory activity of 2,4,6-TIP on Staphylococcus aureus virulence factors, as shown in FIG. 5, 2,4,6-TIP (1, 2, 5, and 10 μg/mL) was shown to decrease hemolytic activity and protease activity in a dose-dependent manner.

### <Experimental Example 4> Cytotoxicity Analysis of Phenol and 2,4,6-TIP

To determine the cytotoxicity of phenol and 2,4,6-TIP, a cabbage seed germination model system and a nematode model (C. elegans) were used.

First, to determine the effect on germination and growth of cabbage (Brassica rapa), cabbage seeds were soaked in sterilized distilled water for 16 hours and rinsed three times with distilled water. To sterilize the seed surface, the seeds were sequentially cultured in 95% ethanol and 3% sodium hypochlorite for 15 minutes at room temperature and rinsed three times with distilled water. Ten seeds per plate were placed on soft agar Murashige-Skoog plates consisting of 0.7% agar and 0.86 g/L Murashige-Skoog (MS) and cultured at room temperature (24° C.) for 7 days. Seed germination rates and plant growth length were measured at the same time every day. Four independent experiments were performed for each concentration of phenol and 2,4,6-TIP (5, 10, 20, and 50 μg/mL).

Additionally, Caenorhabditis elegans (C. albicans strain fer-15 (b26); fem-1 (hc17)) was washed twice with M9 buffer [3 g/L potassium phosphate (KH₂PO₄), 6 g/L dibasic sodium phosphate (Na₂HPO₄), 5 g/L sodium chloride (NaCl), and 1 mM magnesium sulfate (MgSO₄)], and then approximately 30 nematodes were placed in a 96-well plate with M9 buffer (200 μL) containing phenol and 2,4,6-TIP (5, 10, 20, 50, 100, and 200 μg/mL). Afterwards, the plates were cultured at 25° C. for 7 days without stirring. Four independent experiments were performed 3 times. The survival outcomes of the nematodes were determined by their response to LED light for 20-30 seconds after incubation using an iRiS™ digital cell imaging system (Logos Bio Systems).

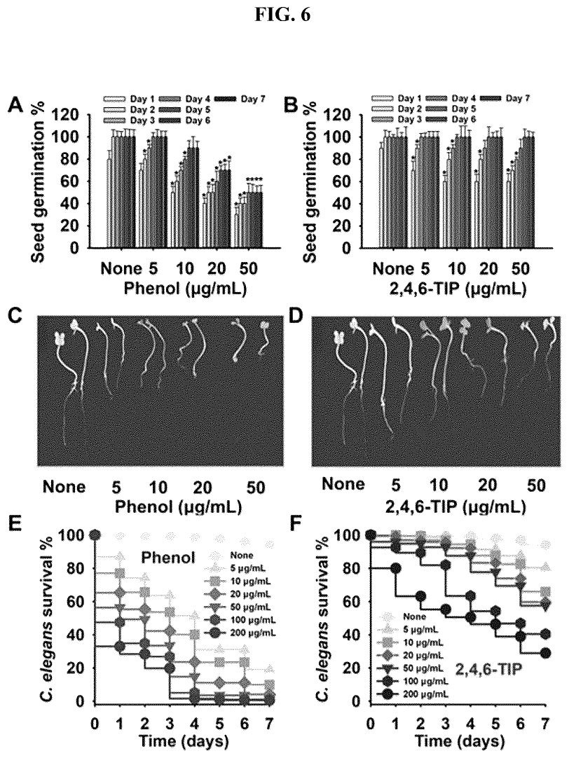

As a result of analyzing cytotoxicity of phenol and 2,4,6-TIP using a cabbage seed germination model, as shown in FIGS. 6A to 6D, phenol decreased the plant germination rate in a concentration-dependent manner at the test concentrations (5, 10, 20, and 50 μg/mL), whereas 2,4,6-TIP did not decrease the plant germination rate after 6 days. The growth rates of the germinated plants also showed similar patterns, finding that 2,4,6-TIP had lower phytotoxicity than phenol.

In addition, as a result of analyzing the cytotoxicity of phenol and 2,4,6-TIP using a nematode model, as shown in FIGS. 6E and 6F, it was determined that 2,4,6-TIP was less toxic to nematodes than phenol. Specifically, it was revealed that approximately 98% of nematodes survived when treated with 5 μg/mL of 2,4,6-TIP for 4 days, whereas 45% of nematodes died when treated with 5 μg/mL of phenol. This means that 2,4,6-TIP at the concentration that shows antibiofilm activity (1-5 μg/mL) has no toxicity to cabbage growth and nematodes.

While a specific part of the present invention has been described in detail above, it is clear for those skilled in the art that this specific description is merely preferred example embodiments, and the scope of the present disclosure is not limited thereby. In other words, the substantial scope of the present disclosure is defined by the appended claims and their equivalents.

What is claimed is:

1. A method of inhibiting a biofilm formation by Staphylococcus aureus or pathogenic microorganisms, comprising:
administering to a subject in need thereof an effective amount of an antibiofilm composition comprising a compound represented by the following Chemical Formula 1 or a pharmaceutically acceptable salt thereof as an active ingredient: 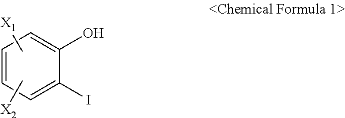
wherein, in the formula,
X₁ and X₂ are the same or different and each independently selected from hydrogen or halogen.

2. The method of claim 1, wherein the compound is a compound represented by the following Chemical Formula 2:

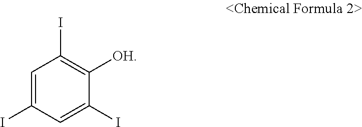

3. The method of claim 1, wherein the Staphylococcus aureus is any one or more selected from the group consisting of Staphylococcus aureus, methicillin-sensitive Staphylococcus aureus (MSSA), methicillin-resistant Staphylococcus aureus (MRSA), and vancomycin-resistant Staphylococcus aureus (VRSA).

4. The method of claim 1, wherein the pathogenic microorganism is selected from the group consisting of Staphylococcus epidermidis, drug-resistant Candida albicans, uropathogenic Escherichia coli, Vibrio parahaemolyticus, and two or more types of microorganisms combined with those and Staphylococcus aureus.

5. The method of claim 1, wherein the composition inhibits a virulence factor of Staphylococcus aureus.

6. The method of claim 1, wherein the composition further comprises any one or more antibiotics selected from the group consisting of vancomycin, gentamycin, streptomycin, ampicillin, and oxytetracycline.

7. A method of inhibiting a growth of Staphylococcus aureus or pathogenic microorganisms, comprising:
administering to a subject in need thereof an effective amount of an antimicrobial composition comprising a compound represented by the following Chemical Formula 1 or a pharmaceutically acceptable salt thereof as an active ingredient: 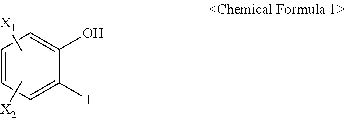
wherein, in the formula,
X₁ and X₂ is the same or different and each independently selected from hydrogen or halogen.

8. A method of preventing or treating an infection caused by a biofilm of Staphylococcus aureus or pathogenic microorganisms,
administering to a subject an effective amount of a pharmaceutical composition comprising a compound represented by the following Chemical Formula 1 or a pharmaceutically acceptable salt thereof as an active ingredient: 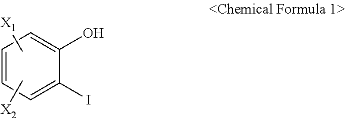
wherein, in the formula,
X₁ and X₂ is the same or different and each independently selected from hydrogen or halogen.

9. The method of claim 8, wherein the infection is any one or more selected from the group consisting of soft tissue infection, septic arthritis, suppurative osteomyelitis, otitis media, pneumonia, postoperative wound infection, bacteremia, endocarditis, sepsis, food poisoning, enteritis, dermatitis, vaginitis, nephritis, cystitis, urethritis, prostatitis, inflammatory diseases caused by urinary catheters or intravenous catheters, and nosocomial infections.

10. The method of claim 9, wherein the soft tissue infection includes cellulitis and pyomyositis.
